(19) United States
(12) Patent Application Publication
(10) Pub. No.: US 2011/0190602 A1
(43) Pub. Date: Aug. 4, 2011

(54) METHOD FOR MEASURING BLOOD OXYGEN AT A FINGER AND FINGER-CLIPPED OXIMETER

(75) Inventors: Weihu Wang, Beijing (CN); Feng Xu, Beijing (CN); Yufei Xie, Beijing (CN); Yan Liu, Beijing (CN)

(73) Assignee: Beijing Choice Electronic Technology Co., Ltd., Beijing (CN)

(21) Appl. No.: 13/075,664

(22) Filed: Mar. 30, 2011

Related U.S. Application Data
(63) Continuation of application No. PCT/CN2010/070420, filed on Jan. 29, 2010.

(30) Foreign Application Priority Data
Jan. 29, 2010 (CN) PCT/CN2010/070420

(51) Int. Cl. A61B 5/00

(52) U.S. Cl. 600/301

(57) ABSTRACT

The present invention provides a method for measuring blood oxygen at a finger, comprising the following steps: 10) measuring blood oxygen content and finger temperature of the human body; 20) giving corresponding prompts according to the blood oxygen content and the finger temperature measured in the step 10). The present invention further provides a finger-clipped oximeter, comprising a control unit, a blood oxygen measurement unit, a finger temperature measurement unit, an output unit and a power management unit. The method for measuring blood oxygen at a finger and the finger-clipped oximeter provided by the present invention can measure the finger temperature of the human body, consider the measured finger temperature as a reference of the measured blood oxygen content of the human body and present it to the user together with the measured blood oxygen content, so as to assist the user in making a more accurate estimation on the blood oxygen content and the health condition of the person under test.

### FIELD OF THE INVENTION

The present invention relates to a technical field of blood oxygen measurement, and particularly relates to a method for measuring blood oxygen at a finger and a finger-clipped oximeter.

### BACKGROUND OF THE INVENTION

With the living standard improving, people pay more attention to his and his family's health, and the amount of oxygen in blood is an important indicator for characterizing the health condition of the human body. In the daily health keeping, it is important for people to acquire his blood oxygen content at any time. For this purpose, a finger-clipped oximeter, suitable for ordinary family members to use, has been developed.

Currently, a finger-clipped oximeter according to the prior art includes an upper case and a lower case. There are components provided in these two cases, for collecting various parameters and performing the processing. When it is used, a person under test only needs to place his finger in a finger container between the two cases to acquire his blood oxygen content so the finger-clipped oximeter is operated in a simple manner. However, as measurement results of the finger-clipped oximeter mentioned above are affected by the finger temperature in practice, a larger error may be produced, and only when the finger temperature is appropriate, the detected blood oxygen content can reflect the level of the blood oxygen content in the human body accurately. For example, when the finger temperature is too low, the periphery of the blood vessel at the finger constricts and the blood flow decreases. At this time, the oximeter may not detect any signal about the blood oxygen, resulting in no corresponding output value; or the oximeter may detect a signal about the blood oxygen, but the measured data can not reflect the level of the blood oxygen content of the entire human body accurately. Therefore, the value of the blood oxygen content of the human body measured in the case that the finger temperature is not known can not reflect the true blood oxygen content of the human body accurately. If the value is used as a determination criterion for the health condition of the person under test, the user may be misled to obtain an inaccurate conclusion, which will lead to a very serious result.

### SUMMARY OF THE INVENTION

To solve the problems mentioned above, the present invention provides a method for measuring blood oxygen at a finger. With this method, the finger temperature, when the blood oxygen content of the human body is measured, can be acquired. Based on the finger temperature, corresponding prompt information is given to assist the user in making a correct judgement of the blood oxygen content of the person under test.

To solve the problems mentioned above, the present invention further provides a finger-clipped oximeter which can also measure the finger temperature and the blood oxygen content of the human body, and gives corresponding prompt information based on the measured finger temperature and the measured blood oxygen content to assist the user in making a correct judgment of the blood oxygen content of the person under test.

So the present invention provides a method for measuring blood oxygen at a finger, which comprises the following steps: 10) measuring blood oxygen content and finger temperature of the human body; 20) giving a corresponding prompt based on the blood oxygen content and the finger temperature measured in the step 10).

Wherein, the step 10) particularly comprises: measuring the finger temperature before measuring the blood oxygen content, or measuring the blood oxygen content and the finger temperature simultaneously, or measuring the finger temperature after measuring the blood oxygen content.

Wherein, the step 10) further comprises: after measuring the finger temperature, determining whether the finger temperature belongs to a finger temperature range suitable for measuring the blood oxygen content.

Wherein, in the step 20), ways to give a corresponding prompt comprise image and/or sound and/or vibration and/or flash.

Wherein, in the step 20), contents to give a corresponding prompt comprise: a value of the blood oxygen content and/or a value of the finger temperature and/or a determination result indicating whether the finger temperature is suitable for the measurement of the blood oxygen content. Preferably, when the finger temperature is suitable for the measurement of the blood oxygen content, a smile face is displayed; or when the finger temperature is not suitable for the measurement of the blood oxygen content, a cry face is displayed.

Moreover, the present invention further provides a finger-clipped oximeter comprising a blood oxygen measurement unit, an output unit and a power management unit, and further comprising a finger temperature measurement unit and a control unit. Wherein, the blood oxygen measurement unit measures the blood oxygen content of the human body according to instructions from the control unit and transmits the value of the blood oxygen content to the control unit, the finger temperature measurement unit measures the finger temperature of the human body according to instructions from the control unit and transmits a value of the finger temperature to the control unit; the control unit instructs the output unit to give a corresponding prompt according to the value of the blood oxygen content from the blood oxygen measurement unit and the value of the finger temperature from the finger temperature measurement unit.

Wherein, the control unit controls an execution order of the blood oxygen measurement unit and the finger temperature measurement unit, which particularly comprises: first executing the blood oxygen measurement unit and then executing the finger temperature measurement unit, or executing the blood oxygen measurement unit and the finger temperature measurement unit simultaneously, or first executing the finger temperature measurement unit and then executing the blood oxygen measurement unit.

Wherein, the control unit determines whether the finger temperature belongs to the finger temperature range suitable for the measurement of the blood oxygen content according to the finger temperature from the finger temperature measurement unit.

Wherein, the output unit comprises a displayer and/or a speaker and/or a vibrator and/or a prompting flash.

Wherein, contents of the corresponding prompt that the control unit instructs the output unit to give comprise: a value of the blood oxygen content and/or a value of the finger temperature and/or a determination result indicating whether the finger temperature is suitable for the measurement of the blood oxygen content.

Advantageous effects that the present invention can bring about are as follows.

The method for measuring the blood oxygen at a finger provided by the present invention can measure the blood oxygen content and the finger temperature of the human body and then give a corresponding prompt according to the blood oxygen content and the finger temperature mentioned above. Therefore, when used to measure the blood oxygen content of the human body, the above method for measuring the blood oxygen is capable of measuring the current finger temperature of the person under test and presenting measurement results to the user, to assist the user in making a correct judgment of the blood oxygen content and thus the health condition of the person under test.

The finger-clipped oximeter provided by the present invention comprises a blood oxygen measurement unit, a finger temperature measurement unit, a control unit, an output unit and a power management unit. Wherein, the control unit may instruct the finger temperature measurement unit to measure the finger temperature of the person under test while instructing the blood oxygen measurement unit to measure the blood oxygen content of the human body, and instructs the output unit to present measurement results to the user according to the measured values of the blood oxygen content and the finger temperature, to assist the user in making a correct judgement of the blood oxygen content and thus the health condition of the person under test.

### BRIEF DESCRIPTION OF THE DRAWINGS

FIG. 1 is a flow chart illustrating a first embodiment of the method for measuring the blood oxygen at a finger provided by the present invention;

FIG. 2 is a flow chart illustrating a second embodiment of the method for measuring the blood oxygen at a finger provided by the present invention;

FIG. 3 is a flow chart illustrating a third embodiment of the method for measuring the blood oxygen at a finger provided by the present invention; and

FIG. 4 is a principal block diagram illustrating one embodiment of the finger-clipped oximeter provided by the present invention.

### DETAILED DESCRIPTION OF THE PREFERRED EMBODIMENTS

In order to enable the persons skilled in the art better understanding the technical solutions of the present invention, the method for measuring the blood oxygen at a finger and the finger-clipped oximeter provided by the present invention will be described in detail, in connection with the figures.

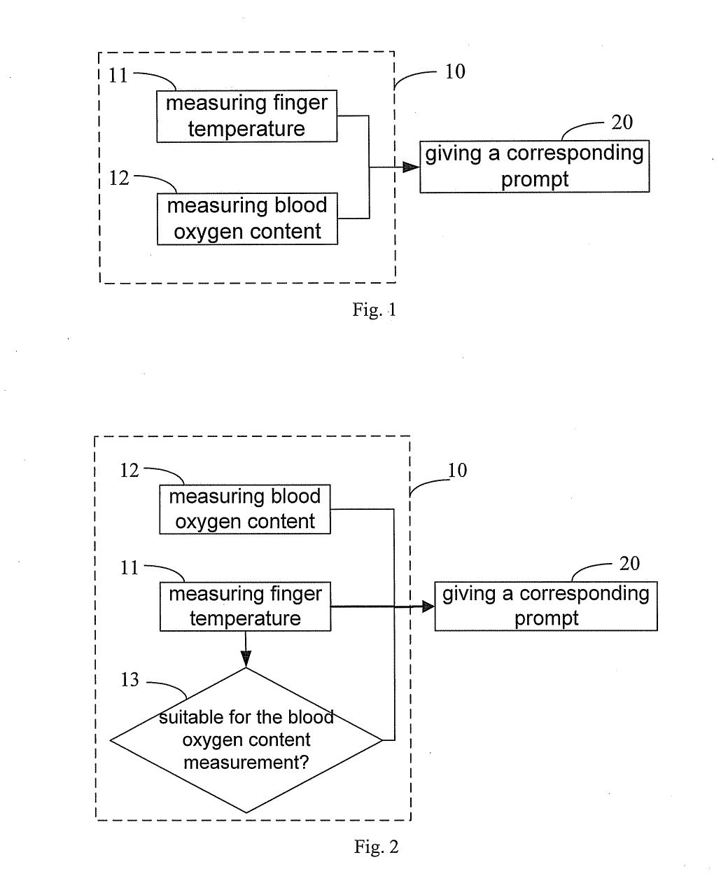

Referring to FIG. 1, a flow chart is illustrated for the method for measuring the blood oxygen at a finger provided by the present invention. The method comprises following steps: 10) measuring the blood oxygen content and the is finger temperature of the human body; 20) giving a corresponding prompt according to the blood oxygen content and the finger temperature measured in the step 10).

Wherein, the step 10) particularly comprises: 11) measuring the finger temperature; 12) measuring the blood oxygen content of the human body. Execution orders of step 11) and the step 12) may comprise: first executing the step 11) and then executing the step 12); or first executing the step 12) and then executing the step 11); or executing the steps 11) and 12) simultaneously. That is, there are multiple choices for the measurement orders of the blood oxygen content and the finger temperature in the step 10). In particular, the finger temperature can be measured before the blood oxygen content is measured, or the blood oxygen content and the finger temperature can be measured simultaneously, or the finger temperature can be measured after the blood oxygen content is measured. Wherein, a following way can be employed to measure the finger temperature: at certain time intervals, measuring the finger temperature several times (for example, at 1-second intervals, continually measuring three times), then calculating an average value of results obtained by measuring several times and using the average value as a final measurement value so as to reduce a possible error of a single measurement.

In the step 20), a corresponding prompt is given according to the blood oxygen content and the finger temperature measured in the step 10), that is, the measured values of the blood oxygen content and/or the finger temperature are presented to the user and the person under test. Here, multiple ways can be employed to achieve the above presentation, for example, employing one or a combination of several ways from image, sound, vibration and flash etc. In particular, an image may be employed to display the measured values of the finger temperature and the blood oxygen content directly, or a sound may be employed to read out the above measured values directly, and so on.

Referring to FIG. 2, a flow chart is illustrated for another embodiment of the method for measuring the blood oxygen at a finger of the present invention. In this embodiment, after measuring the finger temperature in the step 11), the step 10) further comprises step 13): determining whether the finger temperature belongs to a finger temperature range suitable for the blood oxygen content measurement. Here, the finger temperature range suitable for the blood oxygen content measurement is approximately from 20° C. to 40° C., and preferably, the finger temperature range suitable for the blood oxygen content measurement is from 22° C. to 36° C. It should be pointed out that the above mentioned finger temperature range suitable for the blood oxygen content measurement is not limited to that. For example, depending on the actual need for measurement, finger temperature ranges suitable for the blood oxygen content measurement may be further defined with temperature values of 25° C., 26° C., 28° C., 30° C., 31° C., 33° C., 35° C. etc. as endpoints. Of course, the above finger temperature ranges suitable for the blood oxygen content measurement may be further defined with such more accurate temperature values as 24.8° C., 26.2° C., 30.2° C., 31.4° C., 32.8° C., 34.5° C., 35.1° C. etc. as endpoints.

In this embodiment, how to measure the finger temperature is similar to that in the embodiment shown in FIG. 1 and the description thereof is omitted here. Execution orders of the step 11) and the step 12) can also take different execution orders, and the order to execute the step 12) and the step 13) is not required specially. For example, the step 11), the step 12) and the step 13) may be executed in an order of the step 11), the step 12) and the step 13), or in an order of the step 11), the step 13) and the step 12), or in an order of the step 12), the step 11) and the step 13).

The step 20) gives corresponding prompts for values of the blood oxygen content and the finger temperature measured in the step 10), and at the same time, gives a corresponding prompt for the determination result in the above step 13). In particular, an image, a sound, a vibration, a flash etc. may be used to give the corresponding prompts. For example, when the determination result in the step 13) is YES, an image similar to a smile face is displayed, otherwise an image similar to a crying face is displayed; or depending on different finger temperatures, prompt information which indicates that the current finger temperature is normal, on the low side or on the high side is displayed in the displayer respectively; or depending on the different determination result in the step 13), a vibration is generated with a different amplitude and/or frequency and/or duration; or depending on the different determination result in the step 13), an prompt is given with a different sound; or depending on the different determination result in the step 13), an prompt is given with a different flash.

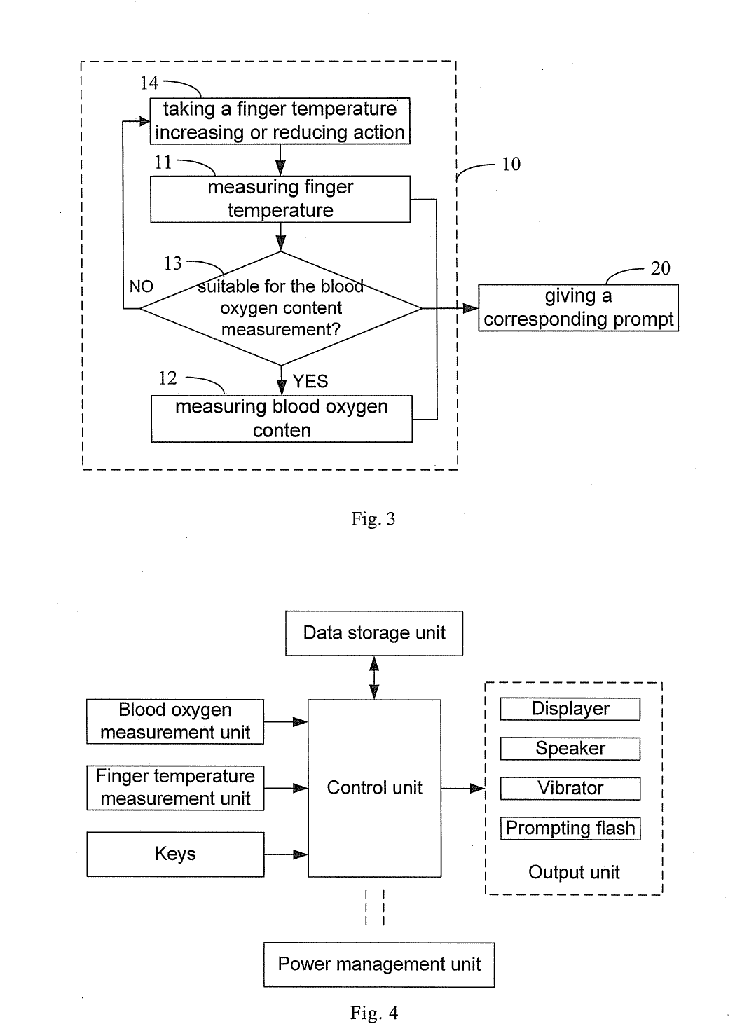

Referring to FIG. 3, a flow chart is illustrated for the third embodiment of the method for measuring the blood oxygen at a finger of the present invention. In this embodiment which is different from the above two embodiments, the blood oxygen content and the finger temperature are measured in a specified order, particularly as follows: 11) measuring the finger temperature of the human body and then turning to the step 13); 13) determining whether the finger temperature measured in the step 11) belongs to the temperature range suitable for the blood oxygen content measurement, if so, executing step 12), and if not so, executing step 14), wherein the temperature range suitable for the blood oxygen content measurement is similar to that in the embodiment shown in FIG. 1 and the description thereof is omitted; 12) measuring the blood oxygen content of the human body; 14) taking a certain temperature increasing or temperature reducing action to make the finger temperature within the temperature range suitable for the blood oxygen content measurement, and then turning to the step 11) so as to re-measure the finger temperature and execute subsequent processes until the determination result in the step 13) is YES, then executing the step 12), after which the measurement process of the step 10) ends.

Wherein, in the step 14), when the finger temperature is too low, the finger of the person under test may become warmed by conventional warming ways such as a hot compression; and when the finger temperature is too high, the finger temperature of the person under test may be decreased by a cold compression.

In the step 20), corresponding prompts are given for information about the values of the blood oxygen content and the finger temperature measured in the above step 10), the determination result in the step 13), etc. in appropriate ways. Contents of the prompts and how to give the prompts are similar to those in the embodiment shown in FIG. 2 and the description thereof is omitted.

It should be pointed out that the above step 14) may be omitted in this embodiment. That is, when the determination result in the step 13) is NO, the step 10) ends. At this time, neither a temperature increasing action or a temperature reducing action is taken for the finger nor the step 12) is executed, but the step 20) is directly executed to give corresponding prompts for the measured finger temperature and the determination result made according to the finger temperature in the step 13). Of course, alternatively, according to the determination result, a prompt may be presented to the user that a certain temperature increasing action or a certain temperature reducing action should be taken so as to make the finger temperature of the person under test within the temperature range required by the blood oxygen content measurement.

In the various embodiments of the method for measuring the blood oxygen at a finger provided by the present invention mentioned above, measuring the finger temperature can be achieved in the following way: first measuring a parameter characterizing the finger temperature and then calculating the value of the finger temperature according to the parameter. Similarly, for the blood oxygen content measurement, a parameter charactering the blood oxygen content of the human body can be measured at a finger and then the value of the blood oxygen content of the human body can be calculated according to the measured parameter.

It should be pointed out that, in the various embodiments of the method for measuring the blood oxygen mentioned above, a step for detecting whether there is a finger to be measured may be added before the step 10), and the subsequent executions will be continued after detect that there is a finger to be measured.

In view of the above, the method for measuring the blood oxygen at a finger provided by the present invention comprises the steps of measuring the blood oxygen content and the finger temperature respectively, and gives corresponding prompts according to the measured blood oxygen content and the finger temperature so that the user can promptly learn the finger temperature while the blood oxygen content is measured, thus making a correct estimation on the accuracy of the measured blood oxygen content. Moreover, the method for measuring the blood oxygen at a finger provided by the present invention further comprises the step of determining whether the measured finger temperature belongs to the temperature range suitable for the blood oxygen content measurement, then presenting the determination result to the user so as to assist the user (especially, the non-professional user) in determining directly whether the current temperature is suitable for the blood oxygen content measurement. Moreover, in a preferred embodiment of the method for measuring the blood oxygen at a finger provided by the present invention, after the above determining step, there is a step of making the finger temperature rise or reduce, and a step of re-executing the measurement of the blood oxygen content of human body until determining that the finger temperature is suitable for the blood oxygen content measurement, so that a more accurate value of the blood oxygen content can be obtained directly. So, in the present invention, the method for measuring the blood oxygen at a finger considers the finger temperature as a reference for determining whether the measured blood oxygen content of the human body is accurate so as to effectively assist the user in making a correct estimation on the actual blood oxygen content and thus the health condition of the person under test.

As another technical solution, the present invention further provides a finger-clipped oximeter. Referring to FIG. 4, a principal block diagram is illustrated for one embodiment of the finger-clipped oximeter provided by the present invention. The finger-clipped oximeter comprises a control unit and a blood oxygen measurement unit, a finger temperature measurement unit, an output unit, a power management unit, a data storage unit and keys which are connected to the control unit.

In particular, the blood oxygen measurement unit measures the blood oxygen content of the human body and transmits a measurement result to the control unit according to instructions from the control unit. The blood oxygen measurement unit comprises two parts, (that is, a top part and a bottom part), wherein one part is a transmitting terminal and other part is a receiving terminal, and these two parts are provided on a transmitting circuit board and a receiving circuit board respectively.

The finger temperature measurement unit measures the finger temperature of the human body and transmits a measurement result to the control unit according to instructions from the control unit. In practice, the finger temperature measurement unit may take a form of a temperature sensor, for example, one or more of a thermocouple, an IR temperature sensor, and a thermistor. In this embodiment, the IR temperature sensor is used, and the accuracy of the sensor at least reaches the medical grade in order that a relatively accurate measurement result can be obtained. Moreover, in practice, the finger temperature measurement unit is often provided near the blood oxygen measurement unit, for example provided near the transmitting terminal or the receiving terminal of the blood oxygen measurement unit.

The control unit controls the execution order of the blood oxygen measurement unit and the finger temperature measurement unit, and instructs the output unit to give corresponding prompts according to a value of the blood oxygen content from the blood oxygen measurement unit and a value of the finger temperature from the finger temperature measurement unit. Moreover, according to the finger temperature from the finger temperature measurement unit, the control unit may also determine whether the current finger temperature belongs to the finger temperature range suitable for the blood oxygen content measurement, that is, determine whether the current finger temperature is suitable for executing the measurement of the blood oxygen content or whether the measured blood oxygen content can characterize a level of the blood oxygen content of the entire human body. The finger temperature range suitable for the blood oxygen content measurement is approximately from 20° C. to 40° C., preferably approximately from 22° C. to 36° C., and in practice, the finger temperature range suitable for the blood oxygen content measurement may be set in the same manner as that in the embodiments of the method for measuring the blood oxygen at a finger. Wherein, as required, the control unit can also instruct the blood oxygen measurement unit and the finger temperature measurement unit to operate in different orders. In particular, the blood oxygen measurement unit is first executed and then the finger temperature measurement unit; or the blood oxygen measurement unit and the finger temperature measurement unit are executed simultaneously; or the finger temperature measurement unit is first executed and then the blood oxygen measurement unit. In practice, the control unit may be a micro controller unit (abbreviated to MCU), such as a single chip.

For example, the output unit may comprise components such as a displayer and/or a speaker and/or a vibrator and/or a prompting flash, which connect to the control unit. According to instructions from the control unit, the output unit presents different contents, and these contents may comprise information such as the value of the blood oxygen content and/or the value of the finger temperature and/or the determination result indicating whether the finger temperature is suitable for the blood oxygen content measurement etc. In particular, the displayer may be used to display the measured data and other associated information directly; the speaker may make sounds according to instructions from the control unit so as to present associated measurement results, or may make an alarm after the measurement result reaches a preset threshold; the vibrator and the prompting flash may present alarm prompts such as vibration and flashing after the measurement result reaches a preset threshold.

The data storage unit is used to store data and information required by the finger-clipped oximeter during operation, can store the value of the blood oxygen content from the blood oxygen measurement unit and the value of the finger temperature from the finger temperature measurement unit according to instructions from the control unit, and all the data stored in the data storage unit can be invoked by the control unit at any time.

The power management unit is used to power all the units and the components.

The keys are used to control the operation of the finger-clipped oximeter. In particular, the keys can be used to turn on and shut down the power, control the execution order of the blood oxygen measurement unit and the finger temperature measurement unit, and set the finger temperature range suitable for the blood oxygen content measurement, etc.

Besides the various units mentioned above, the finger-clipped oximeter provided by the present invention may be further provided with a communication interface, such as a Bluetooth interface and/or a USB interface, to communicate with a PC or a host computer under control of the control unit.

The finger-clipped oximeter provided by the present invention may have following measurement ways.

A first measurement way is as follows.

100) the control unit instructs the blood oxygen measurement unit to measure the blood oxygen content of the human body, and instructs the finger temperature measurement unit to measure the finger temperature;

200) after receiving corresponding data from the blood oxygen measurement unit and the finger temperature measurement unit, the control unit instructs the output unit to give corresponding prompts for the value of the blood oxygen content and the value of the finger temperature.

A second measurement way is as follows.

100) the control unit instructs the blood oxygen measurement unit to measure the blood oxygen content of the human body, and instructs the finger temperature measurement unit to measure the finger temperature;

200) the control unit determines whether the finger temperature belongs to the finger temperature range suitable for the blood oxygen content measurement;

300) the control unit instructs the output unit to give corresponding prompts for the value of the blood oxygen content, the value of the finger temperature and the determination result in the step 200) mentioned above.

A third measurement way is as follows.

100) the control unit instructs the finger temperature measurement unit to measure the finger temperature;

200) the control unit determines whether the finger temperature belongs to the finger temperature range suitable for the blood oxygen content measurement;

300) if the control unit determines that the current finger temperature belongs to the finger temperature range suitable for the blood oxygen content measurement, it instructs the blood oxygen measurement unit to measure the blood oxygen content of the human body and then instructs the output unit to present the value of the blood oxygen content and the value of the finger temperature mentioned above;

400) if the control unit determines that the current finger temperature doesn't belong to the finger temperature range suitable for the blood oxygen content measurement, it will not instruct the blood oxygen measurement unit to measure the blood oxygen content of the human body, but instructs the output unit to prompt the user with the value of the finger temperature mentioned above together with a determination result made according to the finger temperature, so as to prompt the user with that it is inaccurate to measure the blood oxygen content at the current finger temperature. Of course, the output unit may prompt the user in passing with that the user should take a certain temperature increasing or temperature reducing action on the finger of the person under test, so as to make the finger temperature within the temperature range required by the blood oxygen measurement.

In the various measurement ways mentioned above, the finger temperature measurement unit may measure the finger temperature several times at certain time intervals, (for example at 1-second intervals, continually measuring three times, wherein the time interval and the measuring times can be set arbitrarily according to the actual need), and then take the average as a final measured value of the finger temperature so as to reduce a possible error of a single measurement.

In view of the above, the finger-clipped oximeter provided by the present invention comprises the blood oxygen measurement unit which can measure the blood oxygen content of the human body and the finger temperature measurement unit which can measure the finger temperature, and gives corresponding prompts of the blood oxygen content and the finger temperature mentioned above at the output unit, under the control of the control unit. So the user can know promptly the finger temperature when the blood oxygen content is measured, and thus can make a correct estimation on the accuracy of the measured blood oxygen content. Moreover, the control unit in the finger-clipped oximeter provided by the present invention can also determine whether the measured finger temperature belongs to the finger temperature range suitable for the blood oxygen content measurement, and prompt the user with the measured finger temperature together with the determination result, so as to assist the user (especially, the non-professional user) in determining directly whether the current temperature is suitable for the blood oxygen content measurement. Moreover, in a preferred embodiment, when the control unit determines that the current finger temperature is not suitable for the blood oxygen content measurement, it will also prompt the user with the particular temperature increasing or temperature reducing action the user should take, so as to make the operation of the finger-clipped oximeter more humanized. Therefore, the finger-clipped oximeter provided by the present invention considers the finger temperature as a reference for determining whether the measured blood oxygen content of the human body is accurate, so as to effectively assist the user in making a correct estimation on the actual blood oxygen content and thus the health condition of the person under test.

Although several embodiments of the present invention have been described, it should be understood by those skilled in the art, various modifications, improvements and substitutions can be made to the present invention, all of which will fall within the scope of the invention defined by the appended claims.

What is claimed is:

1. A method for measuring blood oxygen at a finger, characterized in that, comprising steps of:
10) measuring blood oxygen content and finger temperature of the human body;
20) giving corresponding prompts according to the blood oxygen content and the finger temperature measured in the step 10).

2. The method for measuring blood oxygen of claim 1, characterized in that, the step 10) particularly comprising: measuring the finger temperature before measuring the blood oxygen content, or measuring the blood oxygen content and the finger temperature simultaneously, or measuring the finger temperature after measuring the blood oxygen content.

3. The method for measuring blood oxygen of claim 2, characterized in that, the step 10) further comprising: after measuring the finger temperature, determining whether the finger temperature belongs to a finger temperature range suitable for the blood oxygen content measurement.

4. The method for measuring blood oxygen of claim 3, characterized in that, in the step 10), the finger temperature range suitable for the blood oxygen content measurement is from 20° C. to 40° C.

5. The method for measuring blood oxygen of claim 3, characterized in that in the step 20), ways of giving corresponding prompts comprising an image and/or a sound and/or a vibration and/or a flash.

6. The method for measuring blood oxygen of claim 3, characterized in that, in the step 20), contents of giving corresponding prompts comprising a value of the blood oxygen content and/or a value of the finger temperature and/or a determination result indicating whether the finger temperature is suitable for the blood oxygen content measurement.

7. The method for measuring blood oxygen of claim 6, characterized in that, in the step 20), when the finger temperature is suitable for the blood oxygen content measurement, displaying with a smile face; or when the finger temperature is not suitable for the blood oxygen content measurement, displaying with a cry face.

8. A finger-clipped oximeter, comprising: a blood oxygen measurement unit, an output unit and a power management unit, characterized in that, further comprising a finger temperature measurement unit and a control unit,
the blood oxygen measurement unit measures blood oxygen content of the human body according to instructions from the control unit and transmits the measured blood oxygen content to the control unit, and the finger temperature measurement unit measures finger temperature of the human body according to instructions from the control unit and transmits the measured finger temperature to the control unit;
the control unit instructs the output unit to give a corresponding prompt according to a value of the blood oxygen content from the blood oxygen measurement unit and a value of the finger temperature from the finger temperature measurement unit.

9. The finger-clipped oximeter of claim 8, characterized in that, the control unit controls execution orders of the blood oxygen measurement unit and the finger temperature measurement unit, which in particular comprise: first executing the blood oxygen measurement unit then the finger temperature measurement unit, or executing the blood oxygen measurement unit and the finger temperature measurement unit simultaneously, or first executing the finger temperature measurement unit then the blood oxygen measurement unit.

10. The finger-clipped oximeter of claim 9, characterized in that, the control unit determines whether the finger temperature belongs to a finger temperature range suitable for the blood oxygen content measurement according to the finger temperature from the finger temperature measurement unit.

11. The finger-clipped oximeter of claim 10, characterized in that, the finger temperature range suitable for the blood oxygen content measurement is from 20° C. to 40° C.

12. The finger-clipped oximeter of claim 10, characterized in that, the output unit comprises a displayer and/or a speaker and/or a vibrator and/or a prompting flash.

13. The finger-clipped oximeter of claim 10, characterized in that, contents of a corresponding prompt that the control unit instructs the output unit to give comprise: the value of the blood oxygen content and/or the value of the finger temperature and/or a determination result indicating whether the finger temperature is suitable for the blood oxygen content measurement.

14. The finger-clipped oximeter of claim 10, characterized in that, further comprising a data storage unit and keys connected to the control unit,
wherein, the data storage unit stores the value of the blood oxygen content from the blood oxygen measurement unit and the value of the finger temperature from the finger temperature measurement unit according to instructions from the control unit;
the keys are used to control execution orders of the blood oxygen measurement unit and the finger temperature measurement unit, and set the finger temperature range suitable for the blood oxygen content measurement.
